(19) United States
(12) Patent Application Publication
(10) Pub. No.: US 2006/0188990 A1
(43) Pub. Date: Aug. 24, 2006

(54) Novel prostate tumor-specific promoter

(75) Inventors: Peter J Kretschmer, San Fancisco, CA (US); Gordon Parry, Oakland, CA (US); Pamela Toy Van Heuit, Moraga, CA (US); Ta-Tung Yuan, Cupertino, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/532,431

(22) PCT Filed: Apr. 16, 2003
(86) PCT No.: PCT/US03/11805

§ 371 Date: Mar. 24, 2006

Related U.S. Application Data
(60) Provisional application No. 60/374,190, filed on Apr. 19, 2002.

(51) Int. Cl. C07H 21/04; C12N 15/86; A61K 48/00

(52) U.S. Cl. 435/456; 424/93.2; 536/23.5

(57) ABSTRACT

This invention provides novel transcriptional regulatory elements from the human TRPM4 (Transient Receptor Potential-Melastatin 4) gene. These promoter and enhancer elements preferentially activate transcription in prostate tumor cells and tissues as compared to other tissues. Methods and compositions are provided to employ TRPM4 promoter elements for prostate tumor-specific expression of therapeutic molecules. Prostate tumor-restricted replicating adenoviral vectors are also provided.

This application claims the benefit of U.S. Provisional Application Ser. No. 60/374,190, filed Apr. 19, 2002, which Is incorporated herein in full by reference.

### FIELD OF INVENTION

This invention provides novel transcriptional regulatory elements from the human TRPM4 (transient receptor potential-melastatin 4) gene. These promoter and enhancer elements preferentially activate transcription in prostate tumor cells as compared to other tissues and cell types. Methods and compositions are provided to employ TRPM4′ promoter elements for prostate tumor-specific expression of therapeutic molecules. Prostate tumor-restricted replicating adenoviral vectors are also provided.

### BACKGROUND OF THE INVENTION

Diseases characterized by uncontrolled proliferation of prostate cells are widespread. Prostate cancer is the second most common cause of cancer death in American males, and benign prostate hyperplasia (BPH) affects 80% of American men over 80. Consequently, much effort has been devoted to treatments that could selectively treat unwanted proliferation of prostate cells without effects on other tissues.

One strategy for targeting therapies to prostate cells takes advantage of prostate-specific gene expression. Several genes, with the prototype being the prostate-specific antigen (PSA) gene, are selectively expressed in the prostate but not other tissues. The promoter elements for several such genes have been cloned, and confer prostate-specific transcription on heterologous genes when re-introduced into prostate cells. A number of therapeutic approaches relying upon prostate-specific transcriptional elements have been envisioned, including therapeutic genes expressed under the control of prostate-specific regulatory sequences and therapeutic viruses whose replication is limited to prostate cells. See U.S. Pat. Nos. 5,648,478, 5,698,443, 5,783,435, 5,830,686, 5,871,726, 5,998,205, 6,051,417, 6,057,299, and 6,136,792.

To date, no genes have been reported to be expressed specifically by prostate tumor cells and tissues. The transient receptor potential-melastatin 4 (TRPM4) is a human protein with channel-like structural motifs homologous to the TRP superfamily and in particular is most homologous to the TRP-melastatin subfamily (Xu et al., Proc. Natl. Acad. Sci. USA (2001), Vol. 98, pp. 10692-97). In humans, the full-length human TRPM4 gene is expressed in a broad variety of tissues and appears in Northern blot analyses as two major mRNA species of ˜4.2 and ˜6.2 kb (see below and Xu et al. (2001), supra). The full-length human TRPM4 gene has been described as SOC-3/CRAC-2 in published PCT patent application No. WO 00/40614, and partial human TRPM4 gene sequences have been disclosed in U.S. Pat. Nos. 6,110,675 and 6,262,245. The present invention relates to our discovery of a portion of this gene that is selectively expressed in prostate tumors and some prostate tumor cell lines. Specifically, a promoter element associated with the TRPM4 gene that confers prostate tumor-specific expression to a reporter gene has been identified.

### BRIEF SUMMARY OF THE INVENTION

The present invention provides compositions and methods for prostate tumor-specific gene expression using the isolated promoter of the TRPM4 gene. In one aspect, the invention provides an isolated polynucleotide comprising a TRPM4 promoter polynucleotide, wherein the TRPM4 promoter polynucleotide is at least 70% identical to SEQ ID NO: 1 over a stretch of at least 70 nucleotides, and confers prostate-specific transcription when operably linked to a heterologous polynucleotide. In another aspect, the TRPM4 promoter polynucleotide is at least 70% identical to SEQ ID NO: 2 over a stretch of at least 70 nucleotides. In some embodiments, the TRPM4 promoter polynucleotides comprise sequences substantially identical to TRPM4 promoter subfragments such as SEQ ID NO: 3 or SEQ ID NO: 4. In some embodiments, the TRPM4 promoter polynucleotides include the transcription initiation elements of the TRPM4 gene, while in other embodiments transcription initiation relies on elements provided by a cis-linked heterologous polynucleotide.

In one embodiment, the TRPM4 promoter polynucleotide is operably linked to a heterologous polynucleotide and may confer prostate tumor-specific gene expression on the heterologous polynucleotide. In some embodiments, the heterologous polynucleotide encodes a therapeutic polypeptide to be expressed in prostate tumor cells, such as a toxin, a prodrug-converting enzyme, a tumor suppressor, a sensitizing agent, an apoptotic factor, an angiogenesis inhibitor, a cytokine, or an immunogenic antigen. In other embodiments, the heterologous polynucleotide encodes a therapeutic polynucleotide such as an antisense RNA molecule or a catalytic RNA molecule.

In one aspect, a TRPM4 promoter polynucleotide of the present invention is comprised in a viral vector, such as a retroviral vector, an adeno-associated viral vector, or an adenoviral vector. In some embodiments, the viral vectors further comprise heterologous polynucleotides operably linked to the TRPM4 promoter polynucleotide. In these embodiments, the polynucleotide may encode a therapeutic polynucleotide such as an antisense RNA molecule or a catalytic RNA molecule, or may encode a therapeutic protein such as a toxin, a prodrug-converting enzyme, a tumor su pressor, a sensitizing agent, an apoptotic factor, an angiogenesis inhibitor, a cytokine, or an immunogenic antigen.

In another aspect the invention provides prostate tumor-restricted replicating adenoviral vectors, which comprise a TRPM4 promoter polynucleotide operably linked to a polynucleotide encoding an adenovirus protein essential for adenoviral replication or propagation. In some embodiments, the adenovirus protein is an adenoviral early gene such as E1a, E1b, E2, or E4. In some embodiments, the replicating adenoviral vector further comprises a heterologous polynucleotide, which may encode a therapeutic polynucleotide such as an antisense RNA molecule or a catalytic RNA molecule, or may encode a therapeutic protein such as a toxin, a prodrug-converting enzyme, a tumor suppressor, a sensitizing agent, an apoptotic factor, an angiogenesis inhibitor, a cytokine, or an immunogenic antigen. Another aspect of the invention provides pharmaceutical compositions comprising the viral vectors of the invention and a pharmaceutically acceptable carrier.

The invention also provides a method of expressing a heterologous polynucleotide in a prostate tumor cell, the method comprising transforming the cell with a TRPM4 promoter polynucleotide operably linked to the heterologous polynucleotide, such that the heterologous polynucleotide is expressed in the prostate tumor cell. In some embodiments, the heterologous polynucleotide encodes a therapeutic polynucleotide such as an antisense RNA molecule or a catalytic RNA molecule, while in other embodiments the heterologous polynucleotide encodes a therapeutic protein such as a toxin, a prodrug-converting enzyme, a tumor suppressor, a sensitizing agent, an apoptotic factor, an angiogenesis inhibitor, a cytokine, or an immunogenic antigen.

### Definitions

The term “TRPM4” refers to a human protein with homology to the transient receptor potential superfamily of channel-like proteins. See Xu et al. (2001), supra.

The term “TRPM4 promoter polynucleotide” refers to a polynucleotide which comprises TRPM4 genomic sequence upstream (5′) of the TRPM4 coding region and activates transcription of a linked polynucleotide in prostate tumor cells. TRPM4 promoter polynucleotides may range from 100 to 5000 nucleotides in length, although in particular embodiments functional TRPM4 promoter polynucleotides may be at least or no more than about 136, 358, 1803, or 2476 nucleotides in length. TRPM4 promoter polynucleotides are generally at least 70% homologous to SEQ ID NO: 1 over a stretch of 70 nucleotides or more. In some embodiments, TRPM4 promoter polynucleotides are at least 75%, 80%, 85%, 90%, 92%, 95%, or 100% homologous to SEQ ID NO: 1 over a stretch of 50, 60, 70, 80, 90, 100, 200, 500, or 1000 nucleotides. TRPM4 promoter polynucleotides contain binding sites for prostate tumor-specific and ubiquitous transcriptional regulatory proteins, and hence activate transcription of linked polynucleotides in prostate tumor cells. TRPM4 promoter polynucleotides confer prostate tumor-specific transcription on linked polynucleotides.

TRPM4 promoter polynucleotides may comprise non-transcribed TRPM4 genomic sequence as well as either TRPM4 introns or exons, or both. In some embodiments, TRPM4 promoter polynucleotides include the TRPM4 transcription initiation sites (collectively referred to as TRPM4 transcription initiation elements) described herein, located from ˜140 to ˜460 nucleotides 5′ of the TRPM4 translation start codon in the mature mRNA. In embodiments where the TRPM4 transcription initiation elements are the only functional initiation elements of the promoter, the natural orientation of the TRPM4 transcription initiation sites, relative to the direction of transcription, should be preserved. In other embodiments, TRPM4 promoter polynucleotides are connected to heterologous TATA boxes and/or transcription initiation sites. When linked to heterologous TATA boxes or transcription initiation sites, TRPM4 promoter polynucleotides act as enhancer elements and may be inserted in either orientation relative to the direction of transcription. Thus, the term “TRPM4 promoter polynucleotide” encompasses polynucleotides comprising the transcription initiation elements of the TRPM4 gene, as well as cis-linked enhancer sequences that yield prostate tumor-specific expression when linked to the transcription initiation elements of a heterologous gene.

The term “prostate tumor-specific expression” or “prostate tumor-specific transcription” means that a polynucleotide is transcribed at a greater rate in prostate tumor cells than in non-tumor prostate cells or non-prostate tumor cells. Thus, a TRPM4 promoter polynucleotide will generally activate transcription of a linked polynucleotide at least 3-fold more efficiently in LNCaP or MDA PCa 2b cells than in BPH-1, Prec, MCF-7 or HepG2 cells, where expression in each case is normalized to the transcription of another polynucleotide linked to the SV40 promoter/enhancer or other constitutive promoter. In certain embodiments, transcription is at least 3-fold, 5-fold, 10-fold, 25-fold or 100-fold more efficient in LNCaP or MDA PCa 2b cells than in BPH-1, Prec, MCF-7 or HepG2 cells. Prostate tumor-specific transcription may result from an increased frequency of transcriptional initiation, an increased rate of transcriptional elongation, a decreased frequency of transcriptional termination, or a combination thereof.

“Transcription initiation elements” refer to sequences in a promoter that specify the start site of RNA polymerase II. Transcription initiation elements may include TATA boxes, which direct initiation of transcription 25-35 bases downstream, or initiator elements, which are sequences located near the transcription start site itself. Eukaryotic promoters generally comprise transcription initiation elements and either promoter-proximal elements, distant enhancer elements, or both. TRPM4 transcription initiation elements may include the transcription initiation sites described herein. Heterologous transcription initiation elements may be obtained from any eukaryotic promoter, although mammalian and viral promoters are preferred sources of heterologous initiation elements.

The term “heterologous polynucleotide” refers to polynucleotides, other than TRPM4 promoter polynucleotides or polynucleotides transcribed from the TRPM4 genomic locus.

A polynucleotide is “expressed” when a DNA copy of the polynucleotide is transcribed into RNA.

A polynucleotide is “operably linked” to a TRPM4 promoter polynucleotide when conjunction of the polynucleotide and the TRPM4 promoter polynucleotide in a single molecule results in prostate tumor-specific transcription. Operable linkage may refer to the conjunction of a TRPM4 promoter polynucleotide to a heterologous polynucleotide to create a prostate tumor-specific expression cassette, or may refer to the conjunction of a TRPM4 promoter polynucleotide to heterologous promoter elements to create a synthetic prostate tumor-specific promoter.

A “prostate cell” is a cell derived from the mammalian prostate gland. In preferred embodiments, prostate cells are derived from the human prostate gland. Prostate cells include normal cells of the prostate, benign prostate hypertrophy (BPH) cells and prostate epithelial cells (Prec), and prostate cancer cells. Prostate tumor cells include prostate cancer cell lines such as DU-145, PC3, MDA PCa 2b, and LNCaP.

A “toxin” is a natural or synthetic polypeptide that results in cell death when expressed. Representative natural toxins include diphtheria toxin, ricin, and Pseudomonas exotoxin.

A “prodrug converting enzyme” is a polypeptide that converts an inactive prodrug into an active drug. Where the active drug is cytotoxic, administration of the prodrug selectively kills cells expressing the prodrug-converting enzyme. Representative prodrug-converting enzymes include herpes simplex virus thymidine kinase, which converts ganciclovir into a DNA chain terminator, and cytosine deaminase, which converts 5-fluorocytosine to 5-fluorouracil, a chemotherapeutic agent.

A “sensitizing agent” is a polypeptide that renders a cell more sensitive to destruction by radiation or a chemotherapeutic agent. Sensitizing agents include proteins that upregulate the apoptotic response to DNA damage and proteins that increase the permeability of cells to a chemotherapeutic agent, such as the thyroid sodium/iodide symporter.

An “apoptotic factor” is a polypeptide that initiates or potentiates apoptosis when expressed in a cell. Representative apoptotic factors include p53, Fas ligand, and bcl-2.

A “cytokine” is a polypeptide that stimulates an immune response by signaling cells of the immune system. Representative cytokines include IL-1, IL-2, IL-12, GM-CSF, and interferons.

An “immunogenic antigen” is a polypeptide that elicits an immune response directed against the cell expressing it. Immunogenic antigens are typically, but not necessarily, foreign polypeptides. Typically, an immunogenic antigen is expressed in a membrane-bound form such that the immune system will mount a cytotoxic response against the cell displaying the antigen.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid is separated from open reading frames that flank the gene and encode other proteins. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res. (1991), Vol. 19, p. 5081; Ohtsuka et al., J. Biol. Chem. (1985), Vol. 260, pp. 2605-08; Rossolini et al., Mol. Cell. Probes (1994), Vol. 8, pp. 91-98). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence that alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

See, e.g., Creighton, Proteins (1984).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three-dimensional structures within a polypeptide. These structures are commonly known as domains. Domains are portions of a polypeptide that form a compact unit of the polypeptide and are typically 15 to 350 amino acids long. Typical domains are made up of sections of lesser organization such as stretches of β-sheet and α-helices. “Tertiary structure” refers to the complete three-dimensional structure of a polypeptide monomer. “Quaternary structure” refers to the three-dimensional structure formed by the non-covalent association of independent tertiary units.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to a nucleotide sequence such as SEQ ID NO: 1), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. (1981), Vol. 2, p. 482; by the homology alignment algorithm of Needleman & Wunsch, J. Mol. Biol. (1970), Vol. 48, pp. 443-53; by the search for similarity method of Pearson & Lipman, Proc. Natl. Acad. Sci. USA (1988), Vol. 85, pp. 2444-48; by computerized implementations of these algorithms (GAP, BESTFIT, FASTA, and TFASTA in the Wisconsin Genetics Software Package, Genetics Computer Group, 575 Science Dr., Madison, Wis.); or by manual alignment and visual inspection (see, e.g., Current Protocols in Molecular Biology (Ausubel et al., eds. 2000 supplement)).

A preferred example of algorithm that is suitable for determining percent sequence identity and sequence similarity are the BLAST and BLAST 2.0 algorithms, which are described in Altschul et al., Nucl. Acids Res. (1977), Vol. 25, pp. 3389-3402 and Altschul et al., J. Mol. Biol. (1990), Vol. 215, pp. 403-10, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nim.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA (1989), Vol. 89, pp. 10915-19) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. USA (1993), Vol. 90, pp. 5873-77). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes (1993), “Overview of principles of hybridization and the strategy of nucleic acid assays.” Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash In 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

### BRIEF DESCRIPTION OF THE DRAWINGS

FIG. 1. Genomic organization of the human TRPM4 gene. The human TRPM4 gene contains twenty-five exons and twenty-four introns. The total length of the gene spanning the amino acid sequence is 54748 bp (position 2386, the ATG translation initiation codon in exon1, to position 57133, the C-terminal amino acid in exon25). The location of the 2476 bp TRPM4 F1R1 promoter polynucleotide (SEQ ID NO: 1) is indicated.

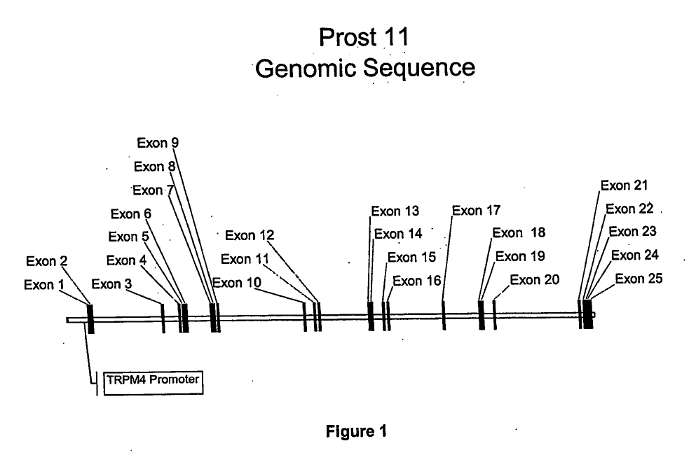

FIG. 2. Transcription initiation sites of the TPRM4 gene mapped by primer extension. The composite nucleotide sequence of the upstream genomic DNA and first exon is shown. Nucleotide positions are numbered in this composite sequence relative to the translation start codon in the first exon, which is designated as +1. The primer extension experiments using RNA from MDA PCa 2b cells indicated a major transcription initiation site at position −151 and a cluster of transcription initiation sites between positions −451 and −431. GT indicates the 5′ position of the first intron.

FIG. 3. Prostate tumor-specific transcriptional activity of a TRPM4 promoter polynucleotide. (Top) TRPM4 promoter constructs used for transient transfections. (Bottom) The TRPM4 promoter constructs (firefly luciferase) were co-transfected with pRLSV40 (Renilla luciferase) into various cell lines as indicated, and luciferase assays were performed 48 hr after transfection. The firefly luciferase activity of each sample was normalized against the Renilla luciferase activity. The normalized luciferase activity of pGL3-Control is defined as 1 (grey bar), and the relative luciferase activity of pGL3-Basic (stippled bar), pGL3bF1R1 (striped bar), pGL3bF1R1-inv (black bar), and PSA promoter (hatched bar) are given as fold increase compared to that of pGL3-Control. Error bars for pF1R1 indicate the standard deviation of the mean of at least three independent transfections.

FIG. 4. Transcriptional activation by TRPM4 promoter polynucleotide fragments in a prostate tumor cell line. (Top) TRPM4 promoter fragments used for promoter mapping experiments. (Bottom) The TRPM4 promoter constructs (pGL3bF1R, pGL3bF1R1-inv, pGL3bF4R1, pGL3bF5R1 and pGL3bF3R1), the PSA promoter construct, pGL3-Control, and pGL3-Basic were co-transfected into MDA PCa 2b prostate tumor cells with pRLSV40 and luciferase activity was normalized to the Renilla luciferase activity as described in FIG. 3. Values are shown compared to that of pGL3-Control, which is defined as 1.

### DETAILED DESCRIPTION OF THE INVENTION

I. Isolation of TRPM4 Promoter Polynucleotides

A. Assaying Prostate Tumor-Specific Transcriptional Activation

1. Assaying Promoter or Enhancer Activity

The present invention provides prostate tumor-specific TRPM4 promoters and enhancers. Accordingly, methods for assaying the prostate tumor-specific transcription induced by TRPM4 promoter polynucleotides are provided herein.

Promoter activity of a TRPM4 promoter polynucleotide is generally assayed by operably linking the TRPM4 promoter polynucleotide to a reporter gene. When inserted into the appropriate host cell, the TRPM4 promoter polynucleotide induces transcription of the reporter gene by host RNA polymerase II. Reporter genes typically encode proteins with an easily assayed enzymatic activity that is naturally absent from the host cell. Typical reporter proteins for eukaryotic promoters include chloramphenicol acetyltransferase (CAT), firefly or Renilla luciferase, beta-galactosidase, beta-glucuronidase, alkaline phosphatase, and green fluorescent protein (GFP). Transcription driven by TRPM4 promoter polynucleotides may also be detected by directly measuring the amount of RNA transcribed from the reporter gene. In these embodiments, the reporter gene may be any transcribable nucleic acid of known sequence that is not otherwise expressed by the host cell. RNA expressed from TRPM4 promoter polynucleotide constructs may be analyzed by techniques known in the art, e.g., reverse transcription and amplification of mRNA, isolation of total RNA or poly A⁺ RNA, northern blotting, dot blotting, in situ hybridization, RNase protection, primer extension, high density polynucleotide array technology and the like.

In addition to reporter genes, vectors for assaying TRPM4 promoter polynucleotide activity also comprise elements necessary for propagation or maintenance in the host cell, and elements such as polyadenylation sequences and transcriptional terminators to increase expression of reporter genes or prevent cryptic transcriptional initiation elsewhere in the vector. Exemplary assay vectors are the pGL3 series of vectors (Promega, Madison, Wis.; U.S. Pat. No. 5,670,356), which include a polylinker sequence 5′ of a luciferase gene. TRPM4 promoter polynucleotide fragments may be inserted into the polylinker sequence and tested for luciferase activity in the appropriate host cell. Assay vectors may also comprise enhancer or transcription initiation sequences, depending on whether the TRPM4 transcription initiation elements are included in the TRPM4 promoter polynucleotide being assayed. Thus, as in Example 4, a TRPM4 promoter polynucleotide including the TRPM4 transcription initiation elements may be inserted into pGL3-Basic, which lacks transcription initiation or enhancer sequences. Here transcription begins at the TRPM4 transcription initiation site(s) and continues through the adjacent luciferase gene. Where the TRPM4 promoter polynucleotide does not include the TRPM4 initiation elements, it may be inserted into an assay vector such as pGL3-Promoter, which includes transcription initiation elements from the SV40 promoter. In such vectors, transcription initiates from a heterologous site but the rate of transcription is increased by the presence of linked TRPM4 enhancer elements.

The ability of a promoter sequence to activate transcription is typically assessed relative to a control construct. In one embodiment, the ability of a TRPM4 promoter polynucleotide to activate transcription is assessed by comparing the expression of a reporter gene linked to a TRPM4 promoter polynucleotide with the expression of the identical reporter gene not linked to TRPM4 promoter polynucleotide sequences. The activity of the promoter is then defined as the fold-increase of reporter gene expression when TRPM4 promoter polynucleotide sequences are present. Thus, in this embodiment, the expression of luciferase is compared between pGL3-Basic and pGL3-Basic with TRPM4 promoter polynucleotide sequences inserted 5′ of the luciferase gene (Example 4). In other embodiments, the activity of a TRPM4 promoter polynucleotide may be compared with that of a known promoter. Thus, the activity of a reporter gene driven by a TRPM4 promoter polynucleotide is compared to the activity of a reporter gene driven by a characterized promoter (e.g., the SV40 promoter/enhancer in pGL3-Control, Promega, Madison, Wis.).

2. Host Systems for Assaying TRPM4 Promoter Activity

While TRPM4 promoter polynucleotides may be assayed for promoter activity using eukaryotic in vitro transcription systems, TRPM4 promoter polynucleotides are typically assayed by transforming them into appropriate host cells, and measuring the expression of reporter genes or other linked polynucleotides.

TRPM4 promoter polynucleotides of the present invention are prostate tumor-specific, activating transcription to a greater extent in prostate tumor cells than in non-tumor prostate cells or non-prostate tumor cells. Accordingly, prostate tumor specificity of a TRPM4 promoter polynucleotide may be assessed by assaying its promoter or enhancer activity in a prostate tumor cell, a non-tumor prostate cell, and a non-prostate cell. Since assays of promoter activity typically compare the expression of a reporter gene in the presence and absence of a TRPM4 promoter polynucleotide, an assay for prostate tumor-specific promoter activity generally requires simultaneous comparison of reporter gene expression in six contexts: the test promoter in a prostate tumor cell, a reference promoter (e.g., lacking TRPM4 sequences) in a prostate tumor cell, the test promoter in a non-tumor prostate cell, the reference promoter in a non-tumor prostate cell, the test promoter in a non-prostate cell, and the reference promoter in a non-prostate cell. Once the promoter activity of the TRPM4 polynucleotide in each cell type is determined by comparing the test promoter and the reference promoter, the prostate tumor specificity of the TRPM4 polynucleotide is calculated by comparing the activity of the test promoter in the prostate tumor cell with its activity in a non-tumor prostate cell and in a non-prostate cell.

One system for assessing TRPM4 promoter activity is transient or stable transfection into cultured cell lines. Assay vectors bearing TRPM4 promoter polynucleotides operably linked to reporter genes can be transfected into any mammalian cell line for assays of promoter activity; for methods of cell culture, transfection, and reporter gene assay see Ausubel et al. (2000), supra; Transfection Guide, Promega Corporation, Madison, Wis. (1998). TRPM4 promoter polynucleotides may be assayed for prostate tumor-specific transcription activity by transfecting the assay vectors in parallel into prostate tumor cell lines, non-tumor prostate cell lines and non-prostate cell lines. Typically, a control vector comprising a second reporter gene driven by a known promoter (e.g., Renilla luciferase driven by the SV40 early promoter/enhancer; pRL-SV40, Promega, Madison, Wis.) is co-transfected along with the assay vector to control for variations in transfection efficiency or reporter gene translation among the prostate tumor, non-tumor prostate and non-prostate cell lines.

Suitable prostate tumor cell lines for assessing prostate tumor-specific transcription are available from the ATCC and include PC-3, MDA PCa 2b, and LNCaP (see U.S. Pat. No. 6,057,299). A preferred cell line is MDA PCa 2b, in which TRPM4 promoter polynucleotides are particularly active (see Example 4). Any readily transfectable mammalian cell line may be used to assay TRPM4 promoter activity in non-tumor prostate cells (e.g., BPH-1 and Prec are such cell lines) and in non-prostate cells (e.g., A549, HT29, SaOs2 and HepG2 are suitable cell lines). Thus, in Example 4, the prostate tumor-specific activity of a 2476 bp TRPM4 promoter polynucleotide is demonstrated by comparing firefly luciferase expression from vectors with and without the 2476 bp TRPM4 promoter fragment in LNCaP, MDA PCa 2b, PC3, DU145, BHP-1, Prec, A549, HepG2, HT29 and SaOs2 cell lines. For each assay, TRPM4 promoter activity is normalized to co-transfected SV40 promoter activity (i.e., pGL3-Contol) to control for variability between the cell lines.

TRPM4 promoter polynucleotide prostate tumor-specific transcription may also be assayed in vivo by employing transgenic animals. Human prostate-specific promoters retain their prostate-specific transcription activity when they are integrated into the genome of transgenic animals (see, e.g., Wei et al., Proc. Natl. Acad. Sci. USA (1997), Vol. 94, pp. 6369-74; Cleutjens et al., Mol. Endo. (1997), Vol. 11, pp. 1256-64; Willis et al., Int. J. Mol. Med. (1998), Vol. 1, pp. 379-86; Wei et al., Int. J. Mol. Med. (1998), Vol. 2, pp. 487-96). Moreover, transgenic animals have been generated that spontaneously develop metastatic prostate cancer (see, e.g., Greenberg et al., Proc. Natl. Acad. Sci. USA (1995), Vol. 92, pp. 3439-43; Gingrich et al., Cancer Res. (1996), Vol. 56, pp. 40964102; Shibata et al., Cancer Res. (1996), Vol. 56, pp. 4894-4903; Masumori et al., Cancer Res. (2001), Vol. 61, pp. 2239-49). Accordingly, transgenic animals with integrated TRPM4 promoter polynucleotides can be used to assay for prostate tumor-specific transcription. In this embodiment, a TRPM4 promoter polynucleotide, linked either to a reporter gene or to native TRPM4 coding sequence, is injected into the embryo of a developing animal (typically a mouse) to generate a transgenic animal. Once integration of the transgene has been verified, such transgenic animals are then crossed with transgenic animals that spontaneously develop metastatic prostate cancer with high frequency. The prostate and non-prostate tissues of the doubly transgenic animals are then assayed for expression of the TRPM4 promoter polynucleotide driven transgene with conventional RNA or protein detection methods known in the art and described herein. Typically, a human TRPM4 polynucleotide is employed, In which case RNA expressed from the transgene may be distinguished from RNA expressed from the endogenous mouse TRPM4 locus by employing appropriate nucleic acid probes that are specific for the human TRPM4 sequence. Alternatively, where the TRPM4 promoter polynucleotide is linked to a reporter gene, tissues of the transgenic animal may be assayed either for reporter gene RNA or for the enzymatic activity of the reporter protein. TRPM4 promoter polynucleotides generally display appropriate prostate tumor-specific regulation regardless of the site of transgene integration; however, TRPM4 promoter constructs may also be flanked by insulator elements (see Bell et al., Science (2001), Vol. 291, pp. 447-50) to ensure complete independence from position effects.

Human TRPM4 promoter polynucleotides display appropriate prostate tumor-specific transcription when integrated into the genome of transgenic animals. However, where it is desirable to assay TRPM4 promoter polynucleotides for in vivo activity in a human cell, nude mice harboring human prostate tumors may be used to test for prostate tumor-specific promoter activity. Procedures for prostate tumor-specific promoter analysis in nude mice with human prostate tumors are described in U.S. Pat. No. 6,057,199. Typically, a nude mouse is injected subcutaneously with an inoculum of human prostate tumor cells (e.g., LNCaP). Following the development of solid tumors, the TRPM4 promoter polynucleotide test construct is injected intravenously in an appropriate delivery medium (e.g., cationic liposomes). Twenty-four hours later, mice are sacrificed and both mouse tissues and the human prostate tumors are assayed for expression of the TRPM4 promoter construct.

B. Isolating TRPM4 Promoter Polynucleotides

1. General Recombinant DNA Methods

This invention relies on routine techniques in the field of recombinant genetics. Basic texts disclosing the general methods of use in this invention include Sambrook et al., Molecular Cloning, A Laboratory Manual (2^{nd} ed. 1989); Kriegler, Gene Transfer and Expression: A Laboratory Manual (1990); and Current Protocols in Molecular Biology (Ausubel et al., eds., 1994)).

For nucleic acids, sizes are given in kilobases (kb), kilobase pairs (kbp) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. (1991), Vol. 22, pp. 1859-62, using an automated synthesizer, as described in Van Devanter et al., Nucl. Acids Res. (1984), Vol. 12, pp. 6159-68. Purification of oligonucleotides is by either native acrylamide gel electrophoresis or by anion-exchange HPLC as described in Pearson & Reanier, J. Chrom. (1983), Vol. 255, pp. 137-49.

The sequence of the cloned genes and synthetic oligonucleotides can be verified after cloning using, e.g., the chain termination method for sequencing double-stranded templates of Wallace et al., Gene (1981), Vol. 16, pp. 21-26.

2. Cloning Methods for the Isolation of TRPM4 Promoter Polynucleotides

In general, the nucleic acid sequences encoding TRPM4 promoter polynucleotides and related nucleic acid sequence homologs are cloned from genomic DNA libraries or isolated using amplification techniques with oligonucleotide primers. For example, TRPM4 promoter polynucleotides are typically isolated from human genomic DNA by PCR amplification with primers that flank the desired promoter polynucleotide. The sequence of these primers can be derived from the TRPM4 sequences disclosed herein or from the genomic sequence of the TRPM4 gene (GenBank accession number AC008891). For example, the 2476 bp TRPM4 F1R1 promoter polynucleotide described herein (SEQ ID NO: 1) may be amplified from human genomic DNA by PCR amplification with the primers F1 (5′-CTCTGTGTCTCTCCTTTGTC-3′) (SEQ ID NO: 5) and R1 (5′-GCTTCCAGACCCGCCCAGA-3′) (SEQ ID NO: 6). The 136 bp TRPM4 F3R1 promoter polynucleotide described herein (SEQ ID NO: 4) may be PCR amplified with the primers F3 (5′-CCTTATCGCGGCCTGGGACC-3′) (SEQ ID NO: 7) and R1 (SEQ ID NO: 6). Any mammalian tissue from which DNA may be easily extracted is a suitable source of genomic DNA for the isolation of mammalian TRPM4 polynucleotides.

TRPM4 promoter polynucleotides can also be isolated from libraries of genomic DNA. Construction of genomic DNA libraries is described in Ausubel et al. (1994), supra. For a genomic library, the DNA is extracted from the tissue and either mechanically sheared or enzymatically digested to yield fragments of about 12-20 kbp. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science (1977), Vol. 196, pp. 180-82. Colony hybridization is carried out as generally described In Grunstein et al., Proc. Natl. Acad. Sci. USA (1975), Vol. 72, pp. 3961-65. Alternatively, genomic DNA libraries are available from various commercial suppliers (e.g., Incyte Genomics, Palo Alto, Calif.; Clontech, Palo Alto, Calif.). Suitable genomic DNA libraries may be prepared using a variety of vectors, including bacteriophages lambda and P1, as well as yeast and bacterial artificial chromosomes.

TRPM4 promoter polynucleotide clones may be identified from genomic libraries either by PCR screening or by hybridization. For PCR screening of genomic libraries, TRPM4 promoter primers are used to amplify ordered pools of genomic libraries (e.g., Easy-to-Screen™ DNA Pools, Incyte Genomics, Palo Alto; CA). Once a positive pool of library DNA is identified by the presence of an amplification product when using the pooled DNA as a template, sub-pools representing fractionations of the original pool are re-screened until a unique library clone is identified.

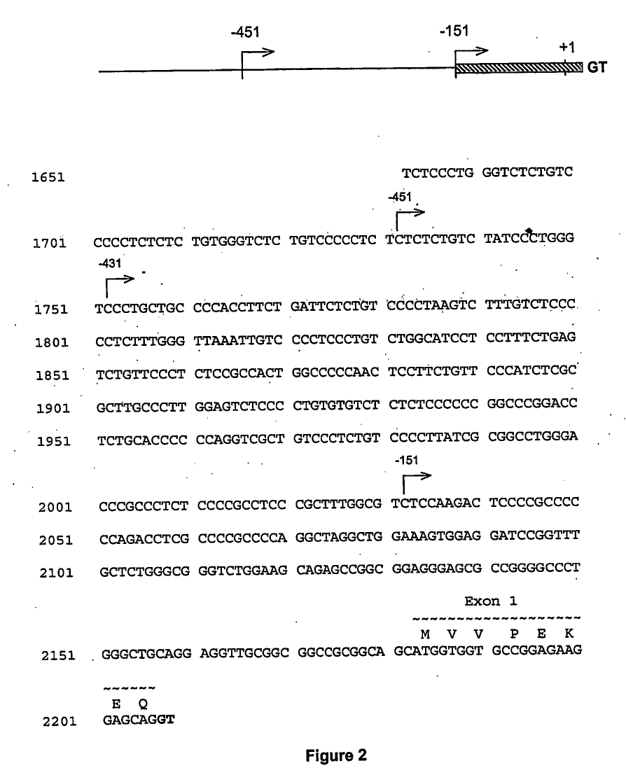

For isolating TRPM4 promoter polynucleotides from genomic DNA libraries by hybridization, individual clones of genomic library DNA are immobilized on a solid substrate such as a nylon filter. Immobilized genomic DNA libraries suitable for screening by hybridization may be constructed as described in Ausubel et al. (1994), supra, or obtained from commercial sources (e.g., Easy-to-Screen™ High-Density Filters, Incyte Genomics, Palo Alto, Calif.). Probes for hybridization screening are labeled fragments of TRPM4 DNA, typically between 100 and 1500 bp in size. A preferred hybridization probe is the 5′ untranslated sequence of the TRPM4 cDNA, upstream of the translation start codon identified in FIG. 2. The cDNA for TRPM4 may be obtained from a cDNA library or other RNA source by amplification with the primers. Once an immobilized genomic sequence containing TRPM4 sequences is identified by hybridization, the corresponding genomic clone is isolated for further analysis. Southern blotting with TRPM4 probes, PCR amplification, or direct DNA sequence analysis may be used to identify the precise TRPM4 promoter polynucleotide isolated, using the sequence of the TRPM4 locus (GenBank accession number AC008891) for reference.

TRPM4 promoter polynucleotides may also be obtained from commercially available bacterial artificial chromosome (BAC) clones. A suitable source for BAC clones is Research Genetics (Huntsville, Ala.). A BAC clone corresponding to GenBank accession number AC008891 is clone #CTD-226J19 from Caltech human BAC clone library D. TRPM4 promoter polynucleotides may be obtained by purifying BAC DNA from a sample of clone #CTD-226J19, amplifying the TRPM4 promoter polynucleotides with primers F1 (SEQ ID NO: 5) and R1 (SEQ ID NO: 6), and isolating the desired DNA fragment by gel electrophoresis.

TRPM4 promoter polymorphic variants, orthologs, and alleles that are substantially identical to TRPM4 promoter polynucleotides can be isolated by screening libraries from the appropriate organism using TRPM4 promoter polynucleotides nucleic acid probes and oligonucleotides under stringent hybridization conditions.

Synthetic oligonucleotides can be also used to construct recombinant TRPM4 promoter polynucleotides for use as probes or for generation of prostate tumor-specific promoters. This method is performed using a series of overlapping oligonucleotides usually 40-120 bp in length, representing both the sense and non-sense (antisense) strands of the gene. These DNA fragments are then annealed, ligated and cloned. Alternatively, amplification techniques can be used with precise primers to amplify a specific subsequence of a TRPM4 promoter polynucleotide.

TRPM4 promoter polynucleotides are typically cloned into intermediate vectors before transformation into prokaryotic or eukaryotic cells for replication and/or expression. These intermediate vectors are typically prokaryotic vectors, e.g., plasmids or shuttle vectors.

C. Functional TRPM4 Promoter Fragments

Once TRPM4 promoter polynucleotides have been isolated, prostate tumor-specific transcriptional activity of a given polynucleotide may be demonstrated by operably linking the promoter polynucleotide to a reporter gene, transfecting the construct into prostate tumor, non-tumor prostate and non-prostate cell lines, and assaying for transcriptional activation as described hereinabove. For example, a PCR fragment of TRPM4 genomic DNA, containing the transcriptional initiation sites and 2476 bp of 5′ upstream sequence from position −2536 to −61 relative to the translation start codon (designated as +1), confers prostate tumor-specific transcriptional activation when operably linked to a reporter gene (Example 4).

Once prostate tumor-specific transcriptional activity has been demonstrated in a TRPM4 promoter polynucleotide, deletions, mutations, rearrangements, and other sequence modifications may be constructed and assayed for prostate tumor-specific transcription in the assays of the invention. Such derivatives of TRPM4 promoter polynucleotides are useful to generate more compact promoters, to decrease background expression in non-prostate tumor cells, to eliminate repressive sequences, or to identify novel prostate tumor-specific transcriptional regulatory proteins. The human and rodent TRPM4 promoter sequences may be compared to identify conserved transcription regulatory elements, including those that confer prostate tumor-specific expression.

TRPM4 promoter sub-fragments and derivatives may be constructed by conventional recombinant DNA methods known in the art. One such method is to generate a series of deletion derivatives within the promoter sequence (Example 5). By comparing the transcriptional activity of a deletion series, the elements that contribute to or detract from prostate tumor-specific transcription may be localized. Based on such analyses, improved derivatives of TRPM4′ promoter polynucleotides may be designed. For example, TRPM4 promoter elements may be combined with prostate-specific or ubiquitous regulatory elements from heterologous promoters to increase the prostate tumor specificity or activity of a TRPM4 promoter polynucleotide.

II. Prostate Tumor-Specific Expression of Therapeutic Molecules

A. General Gene Delivery Methodology

The present invention provides TRPM4 promoter polynucleotides which can be transfected into cells for therapeutic purposes in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. Typically, the operable linkage of a TRPM4 promoter polynucleotide and a therapeutic polynucleotide elicits prostate tumor-specific expression of the therapeutic molecule. The compositions are administered to a patient in an amount sufficient to elicit a therapeutic response in the patient. An amount adequate to accomplish this is defined as “therapeutically effective dose or amount.”

Such gene therapy procedures have been used to correct acquired and inherited genetic defects, cancer, and viral infection in a number of contexts. The ability to express artificial genes in humans facilitates the prevention and/or cure of many important human diseases, including many diseases that are not amenable to treatment by other therapies (for a review of gene therapy procedures, see Anderson, Science (1992), Vol. 256, pp. 808-13; Nabel & Feigner, TIBTECH (1993), Vol. 11, pp. 211-17; Mitani & Caskey, TIBTECH (1993), Vol. 11, pp. 162-66; Mulligan, Science (1993), Vol. 260, pp. 926-32; Dillon, TIBTECH (1993), Vol. 11, pp. 167-75; Miller, Nature (1992), Vol. 357, pp. 455-60; Van Brunt, Biotechnology (1998), Vol. 6, pp. 1149-54; Vigne, Restorative Neurol. Neurosci (1995), Vol. 8, pp. 35-36; Kremer & Perricaudet, British Medical Bulletin (1995), Vol. 51, pp. 31-44; Haddada et al., in Current Topics in Microbiology and Immunology (Doerfler & Böhm eds., 1995); and Yu et al., Gene Therapy (1994), Vol. 1, pp. 13-26).

Delivery of the gene or genetic material into the cell is the first step in gene therapy treatment of disease. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787; and 4,897,355, and lipofection reagents are sold commercially (e.g., Transfectam™ and Lipofectin™). Cationic and neutral lipids that are suitable for efficient receptor-recognition lipofection of polynucleotides include those of Felgner, WO 91/17424 and WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995), Vol. 270, pp. 404-10; Blaese et al, Cancer Gene Ther. (1995), Vol. 2, pp. 291-97; Behr et al., Bioconjugate Chem. (1994), Vol. 5, pp. 382-89; Remy et al., Bioconjugate Chem. (1994), Vol. 5, pp. 647-54; Gao et al., Gene Therapy (1995), Vol. 2, pp. 710-22; Ahmad et al., Cancer Res. (1992), Vol. 52, pp. 4817-20; U.S. Pat. Nos. 4,186,183, 4,217,344,4,235,871, 4,261,975, 4,485,054, 4,501,728,4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kbp of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. (1992), Vol. 66, pp. 2731-39; Johann et al., J. Virol. (1992), Vol. 66, pp. 1635-40; Sommerfelt et al., Virology (1990), Vol. 176, pp. 58-59; Wilson et al., J. Virol. (1989), Vol. 63, pp. 2374-78; Miller et al., J. Virol. (1991), Vol. 65, pp. 2220-24; PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology (1987), Vol. 160, pp. 38-47; U.S. Pat. No. 4,797,368; WO 93/24641; Kotin, Hum. Gene Ther. (1994), Vol. 5, pp. 793-801; Muzyczka, J. Clin. Invest. (1994), Vol. 94, pp. 1351). Construction of recombinant AAV vectors are described in a number of publications, including U.S. Pat. No. 5,173,414; Tratschin et al., Mol. Cell. Biol. (1985), Vol. 5, pp. 3251-60; Tratschin et al., Mol. Cell. Biol. (1984), Vol. 4, pp. 2072-81; Hermonat & Muzyczka, Proc. Natl. Acad. Sci. USA. (1984), Vol. 81, pp. 6466-70; and Samulski et al., J. Virol. (1989), Vol. 63, pp. 3822-28.

In particular, at least six viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., Blood (1995), Vol. 85, pp. 3048-57; Kohn et al., Nat. Med. (1995), Vol. 1, pp. 1017-23; Malech et al., Proc. Natl. Aced. Sci. USA (1997), Vol. 94, pp. 12133-38). PA317/pLASN was the first therapeutic vector used in a gene therapy trial (Blaese et al., Science (1995), Vol. 270, pp. 475-80). Transduction efficiencies of 50% or greater have been observed for MFG-S packaged vectors (Ellem et al., Immunol. Immunother. (1997), Vol. 44, pp. 10-20; Dranoff et al., Hum. Gene Ther. (1997), Vol. 1, pp. 111-23).

Recombinant adeno-associated virus vectors-(rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., Lancet (1998), Vol. 351, pp. 1702-03; Kearns et al., Gene Ther. (1996), Vol. 9, pp. 748-55).

Replication-deficient recombinant adenoviral vectors (Ad) are predominantly used in transient expression gene therapy, because they can be produced at high titer and they readily infect a number of different cell types. Most adenovirus vectors are engineered such that a transgene replaces the Ad E1a, E1b, and E3 genes; subsequently the replication defective vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. (1998), Vol. 9, pp. 1083-92). Additional examples of the use of adenovirus vectors for gene transfer in clinical trials include Rosenecker et al., Infection (1996), Vol. 241, pp. 5-10; Welsh et al., Hum. Gene Ther. (1995), Vol. 2, pp. 205-18; Alvarez et al., Hum. Gene Ther. (1997), Vol. 5, pp. 597-613; Topf et al., Gene Ther. (1998), Vol. 5, pp. 507-13; Sterman et al., Hum. Gene Ther. (1998), Vol. 9, pp. 1083-89.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), Vol. 92, pp. 9747-51, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cell expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3^{rd} ed., 1994)) and the references cited therein for a discussion of how to isolate and culture cells from patients).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, or transdermal application.

Formulations suitable for oral administration can consist of: (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of compositions can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

B. Use of TRPM4 Promoter Polynucleotides for Prostate Tumor-Specific Expression

The TRPM4 promoter polynucleotides of the present invention are useful for specifically expressing therapeutic molecules in prostate tumor cells. Prostate tumor-specific expression of therapeutic molecules may be used, for example, to treat diseases of prostate cancer. Accordingly, therapeutic polynucleotides may be operably linked to TRPM4 promoter polynucleotides and administered to patients to treat prostate diseases or to develop new and improved therapeutics.

Any therapeutic polynucleotide may be operably linked to a TRPM4 promoter polynucleotide. Typically, a TRPM4 promoter polynucleotide is included in an expression cassette and inserted 5′ of the therapeutic polynucleotide to be expressed. TRPM4 promoter polynucleotides may be positioned immediately proximal to the therapeutic polynucleotide although TRPM4 promoter polynucleotides enhancer elements may be positioned anywhere within several kilobases of the therapeutic polynucleotide, including at the 3′ end of the therapeutic polynucleotide and within introns. The ability of a TRPM4 promoter polynucleotide to confer prostate tumor-specific transcription from a given position may be verified by positioning the TRPM4 promoter polynucleotide in the appropriate configuration relative to a reporter gene, and assaying for prostate tumor-specific reporter gene activity as described herein.

Where the TRPM4 promoter polynucleotide includes the TRPM4 transcription initiation elements, the TRPM4 promoter polynucleotide may be linked directly to the polynucleotide encoding a therapeutic molecule without additional sequences. In embodiments where the TRPM4 promoter polynucleotide does not include the TRPM4 transcription initiation elements, additional elements such as a TATA box and transcription initiation sites should be provided. These may either be the transcription initiation elements native to the therapeutic gene, or derived from a heterologous eukaryotic or viral promoter. Additionally, the level of therapeutic gene expression may be increased by including enhancer and polyadenylation sequences from the therapeutic gene or from heterologous genes, so long as the prostate tumor specificity of expression (as measured in the assays of the invention) is maintained.

Vectors for transfecting prostate cells in vitro and in vivo, methods of ensuring sustained expression in prostate cells in vivo, methods of operably linking therapeutic polynucleotides to prostate-specific promoters, methods of targeting vectors to prostate cells in vitro or in vivo, administration routes, and dosages for treatment of prostate disease with therapeutic vectors may be found in, e.g., U.S. Pat. Nos. 5,648,478; 5,698,443; 5,783,435; 5,830,686; 5,871,726; 6,057,299; 6,136,792; and 6,177,410. Gene therapy employing therapeutic molecules expressed by prostate-specific promoters is at an advanced stage of clinical development (see, e.g., Pantuck et al., World J. Urol. (2000), Vol. 18, pp. 143-47). Accordingly, TRPM4 promoter polynucleotides of the present invention can be used for prostate tumor-specific expression of a variety of therapeutic polynucleotides. Therapeutic polynucleotides expressed by TRPM4 promoter polynucleotides are either active themselves (e.g., antisense and catalytic polynucleotides) or encode a therapeutic protein.

1. Antisense and Catalytic Ribonucleotides

One type of therapeutic polynucleotide that may be expressed by TRPM4 promoter polynucleotides is antisense RNA. In such embodiments, the TRPM4 promoter polynucleotide is operably linked to a polynucleotide which, when transcribed by cellular RNA polymerases, is capable of binding to target mRNA. The derivation of an antisense sequence, based upon a cDNA sequence encoding a target protein is described in, for example, Stein & Cohen, Cancer Res. (1988), Vol. 48, pp. 2659-68 and van der Krol et al., BioTechniques (1988), Vol. 6, pp. 958-76. The target protein will generally be an essential cellular gene, a gene required for cell proliferation, or a gene which renders the cell resistant to DNA damage or chemotherapeutic agents. Thus, prostate tumor-specific expression of the antisense molecule preferentially eliminates prostate tumor cells or renders them sensitive to radiation or chemotherapeutic agents. Successful use of prostate tumor-specific antisense expression to treat prostate cancer in vitro and in vivo is described by Lee et al., Anticancer Res. (1996), Vol. 16, pp. 1805-11; Steiner et al., Hum. Gene Ther. (1998), Vol. 9, pp. 747-55; Fan et al., Cancer Gene Ther. (2000), Vol. 7, pp. 1307-14; Eder et al., Cancer Gene Ther. (2000), Vol. 7, pp. 997-1007.

In addition to antisense polynucleotides, ribozymes can be designed to inhibit expression of target molecules. A ribozyme is an RNA molecule that catalytically cleaves other RNA molecules. Accordingly, TRPM4 promoter polynucleotides may be used to express ribozymes specifically in prostate tumor cells by linking a polynucleotide encoding a ribozyme to a TRPM4 promoter polynucleotide. Methods for constructing and using ribozymes to treat prostate cancer in particular are described by Dorai et al., Prostate (1997), Vol. 32, pp. 246-58; Norris et al., Adv. Exp. Med. Biol. (2000), Vol. 465, pp. 293-301. Different kinds of ribozymes have been described, including group I ribozymes, hammerhead ribozymes, hairpin ribozymes, RNase P, and axhead ribozymes (see, e.g., Castanotto et al., Adv. in Pharmacology (1994), Vol. 25, pp. 289-317 for a general review of the properties of different ribozymes). The general features of hairpin ribozymes are described, e.g., in Hampel et al., Nucl. Acids Res. (1990), Vol. 18, pp. 299-304; Hampel et al., European Patent Publication No. 0 360 257 (1990); U.S. Pat. No. 5,254,678. Methods of preparing are well known to those of skill in the art (see, e.g., Wong-Staal et al., WO 94/26877; Ojwang et al., Proc. Natl. Acad. Sci. USA (1993), Vol. 90, pp. 6340-44; Yamada et al., Hum. Gene Ther. (1994), Vol. 1, pp. 39-45; Leavitt et al., Proc. Natl. Acad. Sci. USA (1995), Vol. 92, pp. 699-703; Leavitt et al., Hum. Gene Ther. (1994), Vol. 5, pp. 1115-20; and Yamada et al., Virology (1994), Vol. 205, pp. 121-26).

2. Therapeutic Proteins

A wide variety of therapeutic proteins may be used to treat prostate diseases. Accordingly, the TRPM4 promoter polynucleotides of the present invention may be used to express polynucleotides encoding therapeutic proteins specifically in prostate tumor cells. Therapeutic proteins may be of prokaryotic, eukaryotic, viral, or synthetic origin. Where the therapeutic protein is not of mammalian origin, the coding sequence of the protein may be modified for maximal mammalian expression according to methods known in the art (e.g., mammalian codon usage and consensus translation initiation sites).

Therapeutic proteins which have been successfully employed to treat prostate cell proliferation, and may be operably linked to TRPM4 promoter polynucleotides for prostate tumor-specific expression, include proteins that kill the cell when expressed, such as microbial toxins (Pang, Cancer Gene Ther. (2000), Vol. 7, pp. 991-96) and proteins involved in apoptosis (Li et al., Cancer Res. (2001), Vol. 61, pp. 186-91; Schumacher et al., Int. J. Cancer (2001), Vol. 91, pp. 159-66; Thompson & Yang, Prostate Suppl. (2000), Vol. 9, pp. 25-28; Hyer et al., Mol. Ther. (2000), Vol. 2, pp. 348-58; Griffith et al., J. Immunol (2000), Vol. 165, pp. 2886-94). Prostate cells have been also been targeted by prostate-specific expression of proteins that sensitize prostate cells to therapy. Such proteins may function by converting a prodrug to an active metabolite (e.g., thymidine kinase or cytosine deaminase; for review see Aghi et al., J. Gene Med. (2000), Vol. 2, pp. 148-64), by increasing cell permeability to a therapeutic agent, by restoring hormonal responsiveness, or by rendering the cell more sensitive to radiotherapy or chemotherapeutics. See, e.g., Suzuki et al., Cancer Res. (2001), Vol. 61, pp. 1276-79; O'Keefe et al., Prostate (2000), Vol. 45, pp. 149-57; Cowen et al., Clin. Cancer Res. (2000), Vol. 6, pp. 4402-08; Spitzweg et al., Cancer Res. (2000), Vol. 60, pp. 6526-30; Anello et al., J. Urol. (2000), Vol. 164, pp. 2173-77; Fan et al., Cancer Gene Ther. (2000), Vol. 7, pp. 1307-14; Nielsen, Oncol. Rep. (2000), Vol. 7, pp. 1191-96; Ayala et al., Hum. Pathol. (2000), Vol. 31, pp. 866-70; Boland et al., Cancer Res. (2000), Vol. 60, pp. 3484-92. Other proteins shown to be effective against prostate disease when expressed in prostate cells include proteins that inhibit proliferation or act as anti-oncogenes or tumor suppressors (Shirakawa et al., J. Gene Med. (2000), Vol. 2, pp. 426-32; Tanaka et al., Oncogene (2000), Vol. 19, 5406-12; Okegawa et al., Cancer Res. (2000), Vol. 60, pp. 5031-36; Allay et al., World J. Urol. (2000), Vol. 18, pp. 111-20; Steiner et al., Cancer Res. (2000), Vol. 60, pp. 4419-25), proteins that inhibit angiogenesis (Jin et al., Cancer Gene Ther. (2000), Vol. 7, pp. 1537-42) and proteins that induce an immune response, such as cytokines or foreign antigens (Hull et al., Clin. Cancer Res. (2000), Vol. 6, pp. 4101-09). See also U.S. Pat. No. 6,136,792.

C. Prostate Tumor-Restricted Adenoviruses

Adenoviral vectors are frequently employed for gene therapy of cancer; for review see Zhang, Cancer Gene Ther. (1999), Vol. 6, pp. 113-38. As described hereinabove, TRPM4 promoter polynucleotides can be included in adenoviral vectors for prostate tumor-specific expression of therapeutic genes. However, conventional adenoviral vectors used for gene therapy are usually replication-deficient, lacking one or more of the adenoviral early genes, to prevent infection and lysis of non-malignant tissues. Oncolytic adenoviruses, in contrast, are adenoviruses that will only replicate in a tumor cell. Tumor-specific infection and replication leads to selective lysis of tumor cells. One method of generating tumor- or tissue-restricted oncolytic viruses is to place one or more of the adenovirus early genes (typically E1a, E1b, E2, E4, or combinations thereof) under the transcriptional control of a tumor-specific or tissue-specific promoter. Such vectors are able to selectively replicate in the target tissue (see, e.g., U.S. Pat. No. 5,998,205; Doronin et al., J. Virol. (2001), Vol. 75, pp. 3314-24.

In particular, adenoviral vectors with early genes under the transcriptional control of prostate-specific promoters have been developed. Such vectors can achieve over 10,000:1 selectivity for prostate cells over non-prostate cells, and can eradicate human prostate tumors in mouse models (see Yu et al., Cancer Res. (1999), Vol. 59, pp. 1498-1504; Yu et al., Cancer Res. (1999), Vol. 59, pp. 4200-03). Accordingly, TRPM4 promoter polynucleotides may be operably linked to adenovirus early genes to create adenoviral vectors that are selective for prostate tumor tissue. TRPM4 promoter polynucleotides may be readily substituted for any of the tissue-specific promoters employed in tissue-restricted adenoviral vectors. General methods for construction of recombinant adenoviruses may be found in He et al., Proc. Natl. Acad. Sci. USA (1998), Vol. 95, pp. 2509-14. Construction of prostate-restricted adenoviral vectors, and their administration and use in the treatment of prostate cancer, is described in U.S. Pat. Nos. 5,830,686, 5,871,726, and 5,998,205.

TRPM4 promoter polynucleotides may be linked to any of the adenoviral early genes to yield prostate tumor-limited oncolytic vectors. More than one adenoviral early gene may be driven by a prostate tumor-specific promoter to increase prostate tumor specificity (see Yu et al., Cancer Res. (1999), Vol. 59, pp. 1498-1504; Yu et al., Cancer Res. (1999), Vol. 59, pp. 4200-4203); these additional early genes may be linked to TRPM4 promoter polynucleotides or other prostate tumor-specific promoters. Prostate-restricted adenoviruses may further comprise therapeutic genes as described hereinabove to increase their therapeutic effectiveness. Additional therapeutic genes may be under the control of an unrestricted promoter, or may preferably be under control of a prostate tumor-specific promoter or prostate-specific promoter. Suitable promoters include the prostate tumor-specific TRPM4 promoter and other prostate-specific promoters (e.g., PSA) known in the art.

### EXAMPLES

The following examples are offered to illustrate, but not to limit, the claimed invention.

### Example 1

### Prostate Tumor Specificity of Human TRPM4 Expression

In an Incyte expression database containing EST sequences that are derived from 24 prostate tumor tissue libraries, 3 BPH libraries, 9 normal prostate tissue libraries, and 23 libraries derived from normal tissue adjacent to prostate tumors, a total of 69 TRPM4 cDNA sequences were found, of which 53 (77%) were found in prostate tumor tissue libraries. Thus, TRPM4 is significantly over-expressed in prostate tumor tissue as compared to normal tissue. An Incyte clone (#1512846) containing a 3′ portion of the human TRPM4 cDNA was obtained from Incyte Genomics, Palo Alto, Calif. A TRPM4 fragment was isolated from this clone following restriction enzyme digestion, radioactively labeled with [α-³²P]-dCTP, and used as hybridization probe in northern blot analyses as follows.

To determine the tissue specificity of human TRPM4 expression, total RNA samples were isolated from a variety of normal tissues (prostate, lymph node, pancreas, lung liver, skeletal muscle, breast, kidney, heart, colon—purchased from CLONTECH, Palo Alto, Calif.), tumor tissues (liver, uterus, lung, breast, prostate—purchased from Biochain Inc, Hayward, Calif.) and prostate tumor cell lines (PC3, LNCaP, DU145, MDA PCa 2b). Total RNA was isolated from tissues and cells using RNAeasy™ (QIAGEN Inc., Valencia, Calif.), and were examined for the expression of TRPM4 by northern blot analysis using the above described radioactive human TRPM4 cDNA fragment as probe. TURBOBLOTTER rapid downward transfer systems were used for the northern blot analysis, following procedures recommended by the manufacturer (Schleicher & Schuell, Keene, N.H.).

The integrity of RNA used for this assay was indicated by the detection of GAPDH RNA. Human TRPM4 RNA was detected as ˜4 and ˜6 kb RNA species in all of the above tissues and cell lines except for pancreas, skeletal muscle, heart, liver tumor, uterus tumor, and stomach tumor. Strikingly, a high intensity, small 1.2 kb RNA species was detected only in RNA from human prostate tumors and from the LNCaP cell line. This high intensity band was absent from RNA derived from all of the other tissues and cell lines, including normal prostate tissue. Therefore, although faint expression of the ˜4 and ˜6 kb RNA species was seen in many tissues, as reported by Xu et al. (2001), supra, the small 1.2 kb RNA species appeared to be prostate tumor-specific.

### Example 2

### Isolation of Human TRPM4 Promoter and Genomic Sequences

To isolate human genomic clones encompassing the TRPM4 gene and its promoter sequences, an upstream primer F1 (5′-CTCTGTGTCTCTCCTTTGTC-3′) (SEQ ID NO: 5) and a downstream primer R1 (5′-GCTTCCAGACCCGCCCAGA-3′) (SEQ ID NO: 6) were used in a PCR amplification reaction with human genomic BAC clone #CTD-226J19 (Research Genetics, Huntsville, Ala.) as DNA template. The amplified DNA included 2476 bp of genomic sequence upstream of TRPM4 exon 1 (SEQ ID NO: 1).

### Example 3

### Identification of Transcription Initiation Elements of the TRPM4 Promoter

To identify the transcription initiation elements of the TRPM4 promoter, the start sites of human TRPM4 transcription were determined by primer extension. The primer extension protocol was modified from Sambrook et al. (1989) supra. Briefly, the primer, either R1 (SEQ ID NO: 6) or R2 (5′-ACCCAAAGAGGGGGAGACAAAGACTTAG-3′) (SEQ ID NO: 8), was radiolabeled at its 5′ end with 4 U of T4 polynucleotide kinase and 5 μl of [γ-³²P] ATP (3,000 Ci/mmol, Amersham Pharmacia Biotech, Piscataway, N.J.). The radiolabeled primer (5×10⁴ counts) was mixed with 20 μg RNA from human prostate tissue (CLONTECH, Inc., Palo Alto, Calif.) and incubated with 30 μl of hybridization buffer (80% formamide, 40 mM PIPES, pH 6.4, 1 mM EDTA, pH 8.0, and 0.4 M NaCl) at 85° C. for 10 min, followed by hybridization for 16 hr at 30° C. After ethanol precipitation, reverse transcription was initiated in the reaction buffer containing 30 U of avian myeloblastosis virus reverse transcriptase, 1×RT buffer (Boehringer Mannheim, Indianapolis, Ind.), 1 mM of each dNTP, 1 U of placental RNase inhibitor, and 50 μg/ml actinomycin D, and the reaction was carried out at 42° C. for 90 min. The primer extension product was phenol/chloroform extracted and analyzed on a 6% polyacrylamide gel after ethanol precipitation. The 2476 bp TRPM4 F1R1 promoter polynucleotide (SEQ ID NO: 1) was sequenced using the R1 primer (SEQ ID NO: 6), and used as sequencing ladder.

To identify the transcription initiation sites of TRPM4, total RNA from prostate tissue and two prostate tumor cell lines (LNCaP and MDA PCa 2b) was used for primer extension using the R1 primer (SEQ ID NO: 6). Two major transcription start sites were identified at positions ˜−437 and at −151 (relative to the translation start site ATG, designated as +1; see FIG. 2). To more accurately map the 5′ transcription start site, primer extension was performed with the R2 primer (SEQ ID NO: 8). A cluster of transcription start sites was identified between positions −451 and −431. To exclude the possibility of any DNA-dependent cDNA synthesis, the prostate RNA was treated with RNase or DNase prior to primer extension. No extension products were observed in the RNase treated sample, and the results from the DNase treated sample was the same as the untreated sample. There are no consensus TATA boxes located 25-35 bases upstream of the 2 major transcription initiation sites (see FIG. 2).

### Example 4

### Isolation and Prostate Tumor-Specific Transcriptional Activation of a TRPM4 Promoter Polynucleotide

To demonstrate the prostate tumor-specific activity of TRPM4 promoter polynucleotides, TRPM4 promoter polynucleotides were cloned into reporter plasmids and assayed for prostate tumor-specific transcription. The reporter plasmids pGL3-Basic (which lacks any eukaryotic promoter and enhancer sequences, and has the firefly luciferase gene as a reporter downstream of a multi-cloning site) and pRL-SV40 (which contains a Renilla luciferase reporter gene driven by an early SV40 promoter/enhancer) were obtained from Promega, Inc. (Madison, Wis.). pGL3bF1R1, which contains 2476 bp of TRPM4 genomic sequence upstream of exon 1, was constructed by PCR cloning using the human genomic BAC clone #CTD-226J19 (Research Genetics, Huntsville, Ala.) as DNA template and F1-NheI (5′-CTACTAGCTAGCCTCTGTGTCTCTCCTTTGTC-3′) (SEQ ID NO: 9) and R1-HinDIII (5′-CTAGAAGCTTGCTTGCTTCCAGACCCGCCCAGA-3′) (SEQ ID NO: 10) as primers. The PCR product was gel purified using a gel purification kit (QIAGEN Inc., Valencia, Calif.), digested with NheI and HinDIII and cloned into NheI and HinDIII cut pGL3-Basic. To construct pGL3bF1R1-inv, the restriction enzyme sites in the two PCR primers were switched, resulting in an inverse orientation of the 2476 bp TRPM4 promoter fragment relative to the luciferase gene.

All cell lines were obtained from, and maintained in media recommended by, the American Type Culture Collection (Manassas, Va.). Cells were plated at densities that would result in culture dishes at 80 to 90% confluency in 72 hours. Twenty-four hours post-plating, cells were transfected with test reporter plasmids and FuGENE 6 (Roche Inc., Indianapolis, Ind.) in a 3-to-2 ratio (FuGENE 6:DNA) at a final DNA concentration of 1 μg/ml. pRL-SV40 was routinely co-transfected with the test plasmids as an internal control for transfection efficiency at a final DNA concentration of 0.0125 μg/ml. After 48 hours, the transfected cells were lysed in Passive Lysis Buffer (Promega, Madison, Wis.), and an aliquot of the lysate was measured for luciferase activity using the Dual-Luciferase Reporter Assay System (Promega, Madison, Wis.). The firefly (test) and Renilla (internal control) luciferase activities were measured using a Packard TopCount microplate scintillation counter.

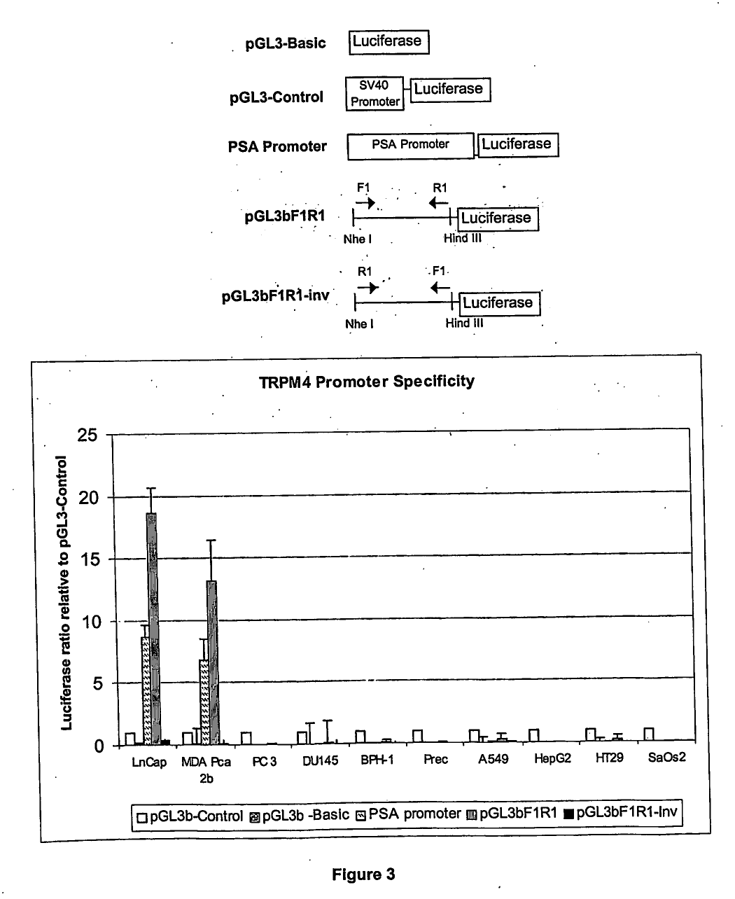

To study the transcriptional regulation of TRPM4, the F1R1 fragment (SEQ ID NO: 1) was cloned in the forward (pGL3bF1R1) or reverse (pGL3bF1R1-inv) orientations into the reporter plasmid pGL3-Basic (FIG. 3). The luciferase activity from the 2476 bp putative promoter fragment was measured 48 hr after transfection into the prostate tumor cell lines, LNCaP, MDA PCa 2b, PC3, and DU145, non-tumor prostate cells BPH-1 and Prec, and the non-prostate cell lines A549, HepG2, HT29, and SaOs2. Transfection efficiency was normalized against the Renilla luciferase activity of the co-transfected pRL-SV40 plasmid (Promega, Madison, Wis.). Insertion of the F1R1 fragment (SEQ ID NO: 1) yielded a minimum of 5-fold increased promoter activity in pGL3bF1R1 compared to the reference level of pGL3-Control (Promega, Madison, Wis.) in MDA PCa 2b and LNCaP cells, but not in the rest of the cells (FIG. 3). Promoter activity was higher in prostate tumor cells (LNCaP and MDA PCa 2b) than in either non-tumor prostate (BPH-1 and Prec) or non-prostate (A549, HepG2, HT29 and SaOs2) cell lines. pGL3bF1R1 displayed more than 100-fold increased luciferase activity versus the pGL3-Basic vector in LNCaP and MDA PCa 2b cells, and the promoter activity was much higher in these prostate tumor cells than in any other cell line tested. In LNCaP and MDA PCa 2b cells, the luciferase activity of the 2476 bp promoter was greater than 10-fold that of the SV40 promoter and enhancer, as measured with pGL3-Control. For comparison, the promoter activity of pGL3bF1R1 in the other cell lines was less than 30% of pGL3-Control. Finally, the orientation specificity of this promoter activity was proven by the construct containing the F1R1 fragment (SEQ ID NO: 1) in the reverse orientation (pGL3bF1R1-inv), which resulted in at least a 50-fold decrease in luciferase activity compared to pGL3bF1R1 in LNCaP and MDA PCa 2b cells.

### Example 5

### Analysis of TRPM4 Prostate Tumor-Specific Regulatory Elements

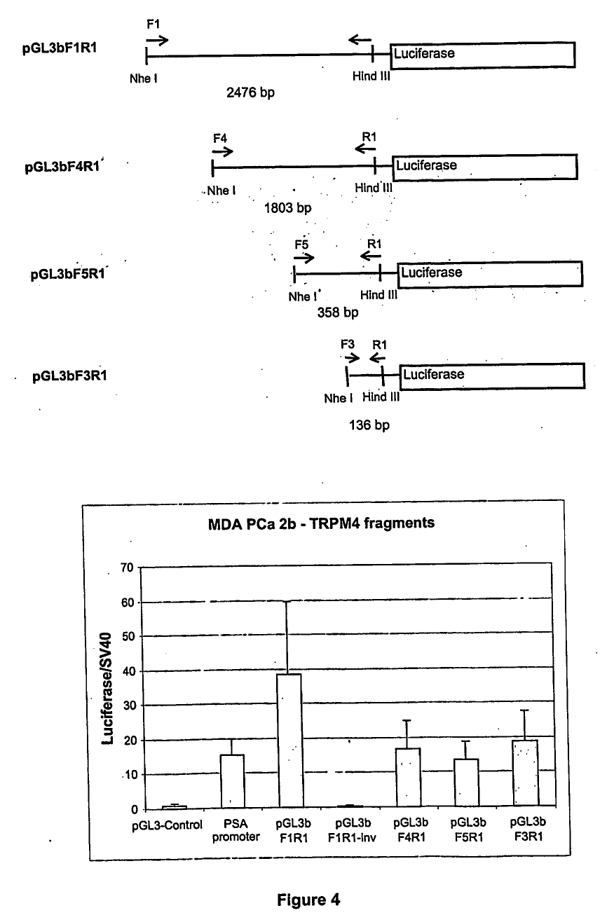

To further dissect the prostate tumor-specific regulatory elements in the 2476 bp TRPM4 F1R1 promoter region, constructs containing serial deletions from the 5′ end of the promoter fragment were generated (see FIG. 4) by PCR amplification using the human genomic BAC clone #CTD-226J19 (Research Genetics, Huntsville, Ala.) as DNA template. pGL3bF4R1, which contains a deletion of 673 bp from the 5′ end of the F1R1 promoter fragment, resulting in a 1803 bp TRPM4 F4R1 promoter polynucleotide (SEQ ID NO: 2) was generated using F4-NheI (5′-CTACTAGCTAGCCCATCACAGAGGGCTGGCAGGAG-3′) (SEQ ID NO: 11) and R1-HinDIII (SEQ ID NO: 10) as PCR primers. pGL3bF5R1, which contains a deletion of 2118 bp from the 5′ end of the F1R1 promoter fragment, resulting in a 358 bp TRPM4 F5R1 promoter polynucleotide (SEQ ID NO: 3) was generated using F5-NheI (5′-CTACTAGCTAGCCCTTCTGATTCTCTGTCCCC-3′) (SEQ ID NO: 12) and R1-HinDIII (SEQ ID NO: 10) as PCR primers. pGL3bF3R1, which the 3′ 136 bp of the F1R1 promoter fragment, resulting in the F3R1 TRPM4 promoter polynculeotide (SEQ ID NO: 4) was created using F3-NheI (5′-CTACTAGCTAGCCCTTATCGCGGCCTGGGACC-3′) (SEQ ID NO: 13) and R1-HinDIII (SEQ ID NO: 10) as PCR primers. Finally, pGL3bF4R5, which contains deletions of 673 bp from the 5′ end and 358 bp from the 3′ end of the F4R1 promoter fragment, was generated using F4-NheI (SEQ ID NO: 11) and R5-HinDIII (5′-CTAGAAGCTTGCTGGGGACAGAGMTCAGAAGG-3′) (SEQ ID NO: 14) as PCR primers. The resulting PCR products were-digested with NheI and HinDIII restriction endonucleases and ligated to NheI and HinDIII cut pGL3-Basic. Each construct was verified by using restriction enzyme digestion.

Each construct was transfected into MDA PCa 2b cells and the resulting luciferase activity was assayed. The deletion of 673 bp from the 5′ end of the F1R1 promoter region resulted in a ˜50% reduction in activity (pGL3bF4R1 in FIG. 4). However, the deletion of 2118 and 2340 bp from the 5′ end of the F1R1 promoter region did not result in any further decrease in luciferase activity (compare pGL3bF5R1 and pGL3bF3R1 to pGL3bF4R1). To investigate whether the 358 bp at the 3′ end of the F1R1 promoter region was essential for promoter activity, this sequence was deleted from the F4R1 promoter fragment and tested (pGL3bF4R5). The resulting TRPM4 sequence did not display any promoter activity, indicating that the 3′ end 358 bp sequence is essential for the promoter activity of the 1803 bp F4R1 promoter region, and, by extension, for the promoter activity of the 2476 bp F1R1 promoter region.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated polynucleotide comprising a TRPM4 (transient receptor potential-melastatin 4) promoter polynucleotide, wherein the TRPM4 promoter polynucleotide is at least 70% identical to SEQ ID NO: 1 over a stretch of at least 70 nucleotides and confers prostate tumor-specific transcription when operably linked to a heterologous polynucleotide.

2. The polynucleotide of claim 1, wherein the polynucleotide comprises a sequence selected from the group consisting of SEQ ID NO: 2, SEQ ID NO: 3, and SEQ ID NO: 4.

3. The polynucleotide of claim 1, wherein the TRPM4 promoter polynucleotide comprises TRPM4 transcription initiation elements.

4. An isolated polynucleotide comprising the TRPM4 promoter polynucleotide of claim 1 operably linked to a heterologous polynucleotide.

5. The polynucleotide of claim 4, wherein the heterologous polynucleotide encodes a polypeptide.

6. The polynucleotide of claim 5, wherein the polypeptide is selected from the group consisting of a toxin, a prodrug-converting enzyme, a tumor suppressor, a sensitizing agent, an apoptotic factor, an angiogenesis inhibitor, a cytokine, and an immunogenic antigen.

7. The polynucleotide of claim 4, wherein the heterologous polynucleotide is selected from the group consisting of an antisense polynucleotide and a catalytic polynucleotide.

8. A viral vector comprising a TRPM4 promoter polynucleotide of claim 1.

9. The viral vector of claim 8, wherein the viral vector is selected from the group consisting of a retroviral vector, an adeno-associated viral vector, and an adenoviral vector.

10. The viral vector of claim 8, wherein the TRPM4 promoter polynucleotide is operably linked to a heterologous polynucleotide.

11. The viral vector of claim 10, wherein the heterologous polynucleotide encodes a polypeptide.

12. The viral vector of claim 11, wherein the polypeptide is selected from the group consisting of a toxin, a prodrug-converting enzyme, a tumor suppressor, a sensitizing agent, an apoptotic factor, an angiogenesis inhibitor, a cytokine, and an immunogenic antigen.

13. The viral vector of claim 10, wherein the polynucleotide is selected from the group consisting of an antisense polynucleotide and a catalytic polynucleotide.

14. An adenovirus vector comprising a TRPM4 promoter polynucleotide of claim 1 operably linked to a polynucleotide encoding an adenovirus polypeptide, wherein the adenovirus polypeptide is essential for adenoviral propagation.

15. The adenovirus vector of claim 14, wherein the polynucleotide encoding the adenovirus polypeptide is selected from the group consisting of the adenovirus E1a, E1b, E2, and E4 genes.

16. The adenovirus vector of claim 14, wherein the adenovirus vector further comprises a polynucleotide selected from the group consisting of an antisense polynucleotide and a catalytic polynucleotide.

17. The adenovirus vector of claim 14, wherein the adenovirus vector further comprises a polynucleotide encoding a polypeptide selected from the group consisting of a toxin, a prodrug-converting enzyme, a tumor suppressor, a sensitizing agent, an apoptotic factor, an angiogenesis inhibitor, a cytokine, and an immunogenic antigen.

18. A composition comprising the adenovirus vector of claim 14 in a pharmaceutically acceptable carrier.

19. A method of expressing a heterologous polynucleotide in a prostate cell, the method comprising transforming the cell with the polynucleotide of claim 4, wherein the heterologous polynucleotide is expressed in the prostate cell.

20. The method of claim 19, wherein the heterologous polynucleotide is selected from the group consisting of an antisense polynucleotide and a catalytic polynucleotide.

21. The method of claim 19, wherein the heterologous polynucleotide encodes a polypeptide selected from the group consisting of a toxin, a prodrug-converting enzyme, a tumor suppressor, a sensitizing agent, an apoptotic factor, an angiogenesis inhibitor, a cytokine, and an immunogenic antigen.


SEQUENCE LISTING (14 sequences)

SEQ ID NO: 1 - DNA, 2476 bp, homo sapien
ctctgtgtctctcctttgtcctttcggtctctgtccaccttcctctggtccttgctgcctggctctggacacccctctgaggctgtctccggagccccctgacccccctctggggccctccctccccattccccagccaatagggtccttcccctcccctctctccagctaaatttactctcagccctgagttattctgggtcagtccccgcctgcctgcctcctgctcctcctcctcccagctggggaggggaccagtgaggggtctctccctggccaggagacggtggccaagggacttgactttgaactaccaacaagctcacgtttggcagctgcaaagacaaaggctagacttttagcaggtttttgggggagcctggggcacctgggggaggcagaagagacttatcagaggggagagactcctgggacggaaggactgggggttcgattgcggggtgtttccagctggaatgatacgtgctggtgagagagtgatgtcagtattgaggccctagaatggggggaaaggaacatggcccccaacacacgtgcccatgacctcctgtccctggaactcagatctgggggcagggactgggctaggccagggctataaatacagctgggaggggtagggggactcaggttacggaggccacagctgtccccatcacagagggctggcaggagacaagtggccttgcccgtctctgtgtgtcagtatttcctactcctcacccttcatgactgcccccactagggtctcctttcctgttcacgggtcctcctctctcttcaattctgtcatctgctctctcagggtccctgtccctcctccatgggattgcctctccctctcactctgggcttctgtcccactcttatcttagtgtcagtcctcccccaagtctgtgtccctctctctcccctaaatctctggcccctcctttctgagttcctgcccttcccccaattctttggtttttgcatccccctctgccccttgcctcagtcaaggtgtctcctccccatctctggcatccacctctctgggtctctgtccccactctctctcagagtctctgtccccctctgtctcagagtctctgtccacctctccctgggtctctgtccccctctctctgggtctctgtccccctctccctgggtctctgtccccctctctctgtggatctctgtccccctctctctgggtctctgttcccctctctctgtgggtctctgtccccctctctctgtggatctctgtccccctctctctgggtctctgttcccctctctctgtgggtctctgtccccctctctctctgggtctctgttcccctctctctgggtctctgtccccctctctcagggtctctgtccccctctgtctcagagtctctgtccccctctctctgggtctctgtcccctctccctgggtctctgtacccctctccgtgggtctctgtcccctctccctgggtctctgtccccccatccctgggtctctgtccccccctctctgggtctctgtccccctctctctgggtctctgtcctcctctctctctgggtctctgttcccctctctctgggtctctgtccccctctctctgggtctctgtccccgtctctctgggtctctgtccccctctctctgggtctctgtcctcctctctctctgggtctctgttcccctctctctgggtctctgtccccctctctctgggtctctgtccccgtctctctgggtctctgtccccctctctctgggtctctgtccccctctctctgggtctctgtcccctctccctgggtctctgtccccctctccgtgggtctctgtcccctctccctgggtctctgtccccccctccctgggtctctgtccccccctctctgggtctctgtccccccctctctgggtctctgtcctcctctccctgggtctctgtccccctctctctgtgggtctctgtcccactctctctgggtctctgtcccactctctctgggtctctgtcccctctccctgggtctctgtccccctctctctgtgggtctctgtccccctctctctctgtctatccctgggtccctgctgccccaccttctgattctctgtcccctaagtctttgtctccccctctttgggttaaattgtcccctccctgtctggcatcctcctttctgagtctgttccctctccgccactggcccccaactccttctgttcccatctcgcgcttgcccttggagtctcccctgtgtgtctctctccccccggcccggacctctgcaccccccaggtcgctgtccctctgtccccttatcgcggcctgggacccgccctctccccgcctcccgctttggcgtctccaagactccccgccccccagacctcgccccgccccaggctaggctggaaagtggaggatccggtttgctctgggcgggtctggaagca

SEQ ID NO: 2 - DNA, 1803 bp, homo sapien
ccatcacagagggctggcaggagacaagtggccttgcccgtctctgtgtgtcagtatttcctactcctcacccttcatgactgcccccactagggtctcctttcctgttcacgggtcctcctctctcttcaattctgtcatctgctctctcagggtccctgtccctcctccatgggattgcctctccctctcactctgggcttctgtcccactcttatcttagtgtcagtcctcccccaagtctgtgtccctctctctcccctaaatctctggcccctcctttctgagttcctgcccttcccccaattctttggtttttgcatccccctctgccccttgcctcagtcaaggtgtctcctccccatctctggcatccacctctctgggtctctgtccccactctctctcagagtctctgtccccctctgtctcagagtctctgtccacctctccctgggtctctgtccccctctctctgggtctctgtccccctctccctgggtctctgtccccctctctctgtggatctctgtccccctctctctgggtctctgttcccctctctctgtgggtctctgtccccctctctctgtggatctctgtccccctctctctgggtctctgttcccctctctctgtgggtctctgtccccctctctctctgggtctctgttcccctctctctgggtctctgtccccctctctcagggtctctgtccccctctgtctcagagtctctgtccccctctctctgggtctctgtcccctctccctgggtctctgtacccctctccgtgggtctctgtcccctctccctgggtctctgtccccccatccctgggtctctgtccccccctctctgggtctctgtccccctctctctgggtctctgtcctcctctctctctgggtctctgttcccctctctctgggtctctgtccccctctctctgggtctctgtccccgtctctctgggtctctgtccccctctctctgggtctctgtcctcctctctctctgggtctctgttcccctctctctgggtctctgtccccctctctctgggtctctgtccccgtctctctgggtctctgtccccctctctctgggtctctgtccccctctctctgggtctctgtcccctctccctgggtctctgtccccctctccgtgggtctctgtcccctctccctgggtctctgtccccccctccctgggtctctgtccccccctctctgggtctctgtccccccctctctgggtctctgtcctcctctccctgggtctctgtccccctctctctgtgggtctctgtcccactctctctgggtctctgtcccactctctctgggtctctgtcccctctccctgggtctctgtccccctctctctgtgggtctctgtccccctctctctctgtctatccctgggtccctgctgccccaccttctgattctctgtcccctaagtctttgtctccccctctttgggttaaattgtcccctccctgtctggcatcctcctttctgagtctgttccctctccgccactggcccccaactccttctgttcccatctcgcgcttgcccttggagtctcccctgtgtgtctctctccccccggcccggacctctgcaccccccaggtcgctgtccctctgtccccttatcgcggcctgggacccgccctctccccgcctcccgctttggcgtctccaagactccccgccccccagacctcgccccgccccaggctaggctggaaagtggaggatccggtttgctctgggcgggtctggaagca

SEQ ID NO: 3 - DNA, 358 bp, homo sapien
ccttctgattctctgtcccctaagtctttgtctccccctctttgggttaaattgtcccctccctgtctggcatcctcctttctgagtctgttccctctccgccactggcccccaactccttctgttcccatctcgcgcttgcccttggagtctcccctgtgtgtctctctccccccggcccggacctctgcaccccccaggtcgctgtccctctgtccccttatcgcggcctgggacccgccctctccccgcctcccgctttggcgtctccaagactccccgccccccagacctcgccccgccccaggctaggctggaaagtggaggatccggtttgctctgggcgggtctggaagca

SEQ ID NO: 4 - DNA, 136 bp, homo sapien
ccttatcgcggcctgggacccgccctctccccgcctcccgctttggcgtctccaagactccccgccccccagacctcgccccgccccaggctaggctggaaagtggaggatccggtttgctctgggcgggtctgga

SEQ ID NO: 5 - DNA, 20 bp, artificial
  primer
ctctgtgtctctcctttgtc

SEQ ID NO: 6 - DNA, 19 bp, artificial
  primer
gcttccagacccgcccaga

SEQ ID NO: 7 - DNA, 20 bp, artificial
  primer
ccttatcgcggcctgggacc

SEQ ID NO: 8 - DNA, 28 bp, artificial
  primer
acccaaagagggggagacaaagacttag

SEQ ID NO: 9 - DNA, 32 bp, artificial
  primer
ctactagctagcctctgtgtctctcctttgtc

SEQ ID NO: 10 - DNA, 33 bp, artificial
  primer
ctagaagcttgcttgcttccagacccgcccaga

SEQ ID NO: 11 - DNA, 35 bp, artificial
  primer
ctactagctagcccatcacagagggctggcaggag

SEQ ID NO: 12 - DNA, 32 bp, artificial
  primer
ctactagctagcccttctgattctctgtcccc

SEQ ID NO: 13 - DNA, 32 bp, artificial
  primer
ctactagctagcccttatcgcggcctgggacc

SEQ ID NO: 14 - DNA, 33 bp, artificial
  primer
ctagaagcttgctggggacagagaatcagaagg